(19) United States
(12) Patent Application Publication
(10) Pub. No.: US 2021/0231598 A1
(43) Pub. Date: Jul. 29, 2021

(54) DETECTION OF 1, 5-ANHYDROGLUCITOL (1, 5-AG) IN SALIVA

(71) Applicant: NANOBIO SYSTEMS INC., Marblehead, MA (US)

(72) Inventors: Eve F. FABRIZIO, Avon, OH (US); Bindi PATEL, Westlake, OH (US); Pamela HENDRIX, Sandusky, OH (US); Kraig HOLLER, Fairview Park, OH (US); Seyamak KEYGHOBAD, Marion, MA (US)

(21) Appl. No.: 17/054,793

(22) PCT Filed: Apr. 9, 2019
(86) PCT No.: PCT/US2019/026476

§ 371 Date: Nov. 12, 2020

Related U.S. Application Data
(60) Provisional application No. 62/723,111, filed on Aug. 27, 2018.
(60) Provisional application No. 62/673,414, filed on May 18, 2018.
(60) Provisional application No. 62/820,455, filed on Mar. 19, 2019.

(51) Int. Cl. G01N 27/327; C12Q 1/00

(52) U.S. Cl. CPC G01N 27/3271; C12Q 1/005; G01N 2800/042; G01N 2400/00; G01N 27/3278

(57) ABSTRACT

A system can facilitate detection of 1, 5-anhydroglucitol (1, 5-AG) present in a saliva sample. The saliva sample can be placed in contact with a portion of a sensing device that includes a 1, 5-AG sensor. An indicator of a concentration of 1, 5-AG in the saliva sample can be detected by the 1, 5-AG sensor. The 1, 5-AG sensor can send the indicator to a computing device that includes a processing unit. The processing unit can execute instructions stored in non-transitory memory to receive the indicator; measure an amount of 1, 5-AG in the saliva sample based on the indicator; and provide an output related to the amount of 1, 5-AG in saliva sample. The amount of 1, 5-AG in the saliva sample can be used as a diagnostic property for a subject who produced the saliva sample.

### RELATED APPLICATIONS

This application claims priority to U.S. Provisional Application Ser. No. 62/673,414, filed May 18, 2018, and entitled “ELECTROCHEMICAL SENSOR SYSTEM TO DETERMINE 1, 5-ANHYDROGLUCITOL LEVELS IN HUMAN SALIVA”. This application also claims priority to U.S. Provisional Application Ser. No. 62/723,111, filed Aug. 27, 2018, and entitled “DETECTION OF 1, 5-ANHYDROGLUCITOL (1, 5-AG) IN SALIVA AS AN INDICATOR OF GLYCEMIC VARIABILITY AND GLUCOSE DYSREGULATION”. This application also claims priority to U.S. Provisional Application Ser. No. 62/820,455, filed Mar. 19, 2019, and entitled “SALIVA COLLECTION AND TREATMENT”. These provisional applications are hereby incorporated by reference in their entirety for all purposes.

### TECHNICAL FIELD

The present disclosure relates generally to detecting 1, 5-anhydrogluticol (1, 5-AG) in saliva, and, more specifically, to systems and methods that facilitate detection of an amount of 1, 5-AG present in a saliva sample.

### BACKGROUND

Diabetes represents a growing health problem in the U.S. and throughout the world. More than 100 million U.S. adults are now living with diabetes, pre-diabetes, or insulin resistance and metabolic syndrome. Currently, the only way to identify or monitor diabetes, pre-diabetes, and/or insulin resistance and metabolic syndrome is through laboratory blood tests. For example, the hemoglobin A1c test (A1C) is the gold standard laboratory blood test to provide a picture of average blood glucose control for the past 2 to 3 months (the time it takes for red blood cells to turn over in the blood stream); however, due to several issues (including the poor sensitivity of A1C), A1C cannot be used alone as a diagnostic. Instead, A1C must be used in connection with a blood glucose test to increase diagnostic reliability. While A1C does not vary depending on food consumption, the blood glucose test is highly variable depending on food consumption.

A glucose analog, 1, 5-anhydrogluticol (1, 5-AG), can be used as an alternative to identify or monitor diabetes, pre-diabetes, and/or insulin resistance and metabolic syndrome. 1, 5-AG is a naturally occurring monosaccharide that is a non-metabolizable glucose analog (only differing in chemical structure from glucose because 1, 5-AG is missing a C-1 hydroxyl group). Advantageously, 1, 5-AG is unaffected by food consumption, so measurements of 1, 5-A-G can reflect glucose control over the past week. However, conventional methods for measuring 1, 5-AG require blood to be drawn and a laboratory test using biochemical assays, making such conventional methods for measuring 1, 5-AG unsuitable for point of care diagnostics.

### SUMMARY

The present disclosure provides a non-invasive point of care diagnostic that can identify 1, 5-anhydrogluticol (1, 5-AG) levels in saliva, not the traditional blood. The non-invasive point of care diagnostic can be used as an at-home screening tool to identify and/or monitor diabetes, prediabetes, and/or insulin resistance and metabolic syndrome.

In one aspect, the present disclosure can include a system that can detect an amount of 1, 5-AG present in a subject's saliva. The system includes a sensing device that includes a 1, 5-AG sensor, which can be configured to be placed in contact with a saliva sample and to detect an indicator of a concentration of 1, 5-AG in the saliva sample. The system also includes a computing device that includes a processing unit configured to: receive the indicator of the concentration of 1, 5-AG in the saliva sample; measure an amount of 1, 5-AG in the saliva sample based on the indicator of the concentration of 1, 5-AG in the saliva sample; and provide an output related to the amount of 1, 5-AG in the saliva sample. The amount of 1, 5-AG in the saliva sample can be used a diagnostic indicator. For example, the diagnostic indicator can be related to diabetes, pre-diabetes, and/or insulin resistance and metabolic syndrome.

In another aspect, the present disclosure can include a method for detecting an amount of 1, 5-AG present in a subject's saliva. A 1, 5-AG sensor can be placed in contact with a saliva sample. The 1, 5-AG sensor can detect an indicator of 1, 5-AG. The indicator of 1, 5-AG can be sent to a computing device. The computing device can measure the amount of 1, 5-AG in the saliva sample based on the indicator. The amount of 1, 5-AG in the saliva sample can be used as a diagnostic indicator. For example, the diagnostic indicator can be related to diabetes, pre-diabetes, and/or insulin resistance and metabolic syndrome.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram showing an example of a system that can be used to detect the presence of 1, 5-anhydrogluticol (1, 5-AG) in a saliva sample;

FIGS. 2 and 3 are block diagrams showing example implementations of the sensing device shown in FIG. 1;

FIG. 4 is a block diagram showing an implementation of the processing of the computing device shown in FIG. 1;

FIG. 5 is a process flow diagram showing an example method for detecting the presence of 1, 5-AG in a saliva sample;

FIG. 6 is a process flow diagram showing an example method for determining an amount of 1, 5-AG in a saliva sample;

FIG. 7 is a process flow diagram showing an example method for determining a level of glycemic variability based on amounts of 1, 5-AG detected in saliva samples;

FIG. 8 is a block diagram showing an example group of devices that can be used to detect the presence of 1, 5-AG in a saliva sample;

FIG. 9 is a plot showing an increase in current due to an increasing amount of 1, 5-AG measured as a function of time;

FIG. 10 is a plot showing a linear current response for increasing 1, 5-AG concentration in saliva;

FIG. 11 is a plot showing a patient's weekly 1, 5-AG readings from 9 Jul. 2018 until 8 Aug. 2018;

FIG. 12 is a plot showing a patient's weekly blood glucose (BG) readings from 9 Jul. 2018 until 8 Aug. 2018; and

FIG. 13 is a plot showing measurement of blood glucose and 1, 5-AB for a subject over two days.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “sensor” refers to a device that detects or measures a physical property and records, indicates, or otherwise responds to the measured physical property. As an example, the sensor can detect or measure the physical property (current) produced based on the concentration of the analyte in the sample by an electrochemical method, like amperometry, coulometry, voltammetry, etc. The sensor can determine the presence of an analyte in a sample either directly or indirectly (through detecting a secondary effect of the analyte).

As used herein, the term “sample” refers to a volume of a specimen (e.g., a fluid, colloid, suspension, etc.) taken for testing or analysis. For example, the sample can include a biofluid, like saliva.

As used herein, the term “analyte” refers to a substance whose chemical constituents are being identified and measured. An example of an analyte is 1, 5-anhydrogluticol (1, 5-AG).

As used herein, the term “diagnostic indicator” can refer to something that serves to identify a particular disease or characteristic, such as pre-diabetes, insulin resistance and metabolic syndrome, diabetes, and the like.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

The present disclosure relates generally to detecting 1, 5-anhydrogluticol (1, 5-AG) in saliva. An amount of 1, 5-AG present in a subject's saliva can be detected by a 1, 5-AG sensor. The 1, 5-AG sensor can be configured to be placed in contact with the subject's saliva and to detect an indicator of a concentration of 1, 5-AG in the patient's saliva. The 1, 5-AG sensor can send the indicator of the concentration of 1, 5-AG to a computing device that includes a processor, which can at least measure an amount of 1, 5-AG in the subject's saliva based on the indicator of the concentration of 1, 5-AG in the subject's saliva; and provide an output related to the amount of 1, 5-AG in the subject's saliva. The amount of 1, 5-AG in the subject's saliva can be used a diagnostic indicator. For example, the diagnostic indicator can be related to diabetes, pre-diabetes, and/or insulin resistance and metabolic syndrome.

Unlike conventional solutions, the present disclosure provides a non-invasive diagnostic that can identify 1, 5-AG levels in saliva. For example, a drop in 1, 5-AG can be identified at least 24 hours after a postprandial hyperglycemic event (e.g., a meal). The non-invasive diagnostic can be used as an at-home and/or point of care screening tool for diabetes, prediabetes, and/or insulin resistance and metabolic syndrome (e.g., the 1, 5-AG levels can indicate that a subject exhibits postprandial hyperglycemia, which is a symptom of insulin resistance). Monitoring and tracking 1, 5-AG on a daily or weekly basis provides timely feedback on how the subject is controlling blood glucose, especially after eating, allowing the subject to gain better understanding of how diet and exercise affect hyperglycemia and/or glucose variability, helping the subject ultimately minimize hyperglycemia and/or glucose variability.

### III. Systems

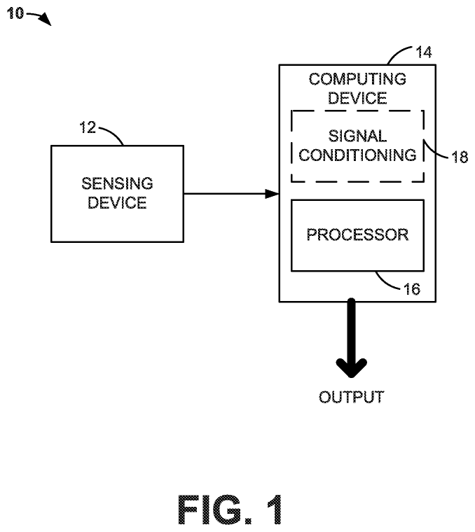

One aspect of the present disclosure can include a system 10 (FIG. 1) that can detect the presence of 1, 5-anhydrogluticol (1, 5-AG) in a saliva sample. The system 10 provides an easy to use, noninvasive point-of-care diagnostic that can be used as an at-home and/or point of care screening tool for diabetes, prediabetes, and/or insulin resistance and metabolic syndrome. For example, when used as a point-of-care diagnostic, the presence of 1, 5-AG can be detected in the saliva sample a time (e.g., 24 hours or more) after the patient has experienced an extended glucose spike (e.g., with or after eating). When an abnormal amount of 1, 5-AG is detected in the saliva sample, the patient can be indicated as exhibiting postprandial hyperglycemia. Alternatively, the system 10 can be used to screen patients who may be exhibiting hyperglycemia (and corresponding high blood sugar readings) due to a medical condition like an infection or cancer, but not exhibiting diabetes.

The amount of 1, 5-AG detected in the saliva sample can be used as a diagnostic indicator. For example, when the amount of 1, 5-AG is present in an amount greater than 10 μg/dL (or a “high” level), the patient can be diagnosed as normal. When the amount of 1, 5-AG is present in an amount less than 10 μg/dL, the patient can be diagnosed as exhibiting an abnormal reading (or a “low” level). The high level and/or the low level can be used as diagnostic indicators. In some instances, the low level can correspond to a diagnosis of the patient exhibiting glycemic variability. When the patient is exhibiting glycemic variability with a likelihood of hyperglycemia, the patient can have other test results (e.g., from a blood glucose reading, an H1c reading, a salivary glucose reading, etc.) to complete a diagnosis of diabetes, prediabetes, and/or insulin resistance and metabolic syndrome. In other instances, the low level can be used as an impetus to recommend further testing to isolate the cause of the low reading. In still other instances, the high level can be used to identify the patient as “normal” (exhibiting minimal glycemic variability, not exhibiting diabetes, pre-diabetes, and/or insulin resistance and metabolic syndrome).

As shown in FIG. 1, the system 10 can include a sensing device 12 and a computing device 14. The sensing device 12 can be placed in contact with a saliva sample taken from a patient and can detect an indicator of a concentration of 1, 5-AG in the saliva sample. The computing device 14 includes a processing unit 16, which can at least receive the indicator of the concentration of 1, 5-AG in the saliva sample; measure an amount of 1, 5-AG in the saliva sample based on the indicator of the concentration of 1, 5-AG in the saliva sample; and provide an output related to the amount of 1, 5-AG in the saliva sample. The amount of 1, 5-AG in the saliva sample can be used as a diagnostic indicator, as noted above.

The system 10 can include a mechanism to provide the saliva sample to the sensing device 12 for analysis and detection of 1, 5-AG. In some instances, the sensing device 12 can be put into an environment with a previously collected sample. In other instances, the sensing device can be put into an environment to collect the sample. In either instance, the sample can be preprocessed (e.g., using a filter device) to prepare the sample for exposure to the sensing device 12. For example, the sample can cover at least a portion of the sensing device. The sensing device 12 can define a volume of the saliva sample necessary for analysis.

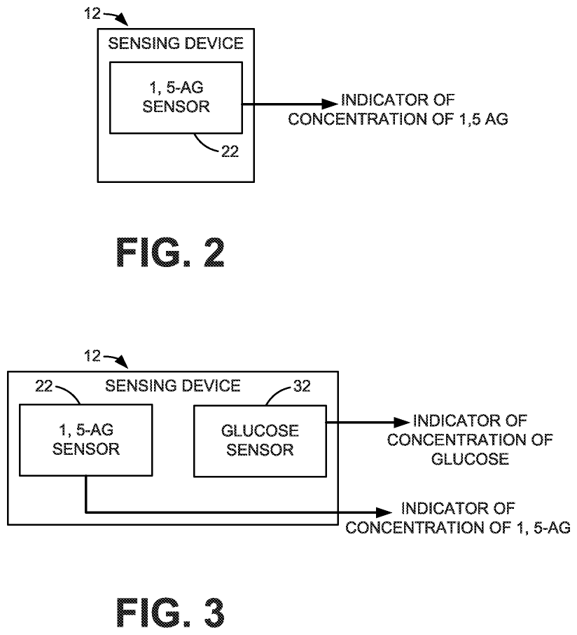

The sensing device 12 can include a 1, 5-AG sensor 22, as shown in FIG. 2. The 1, 5-AG sensor can be placed in contact with a saliva sample and can detect the presence of 1, 5-AG in the saliva sample (e.g., by detecting an indicator of the 1, 5-AG). The detection can be via an electrochemical method (with a sensitivity to detect 1, 5-AG at concentrations as low as 0 μg/mL and as high as at least 50 μg/mL) so that the indicator can be a current produced based on the concentration of 1, 5-AG in the saliva sample. For example, the electrochemical method can employ amperometry, coulometry, voltammetry, or the like. The sensing device 12 can send the indicator of the 1, 5-AG to the computing device 14, which can measure the amount of 1, 5-AG in the saliva sample based on the indicator of the 1, 5-AG. In other examples, the detection can be via an impedance sensing method.

In some instances, the 1, 5-AG sensor 22 can determine the presence of 1, 5-AG directly, such that the detected 1, 5-AG can be transformed into a measurable signal that can be related to the presence of 1, 5-AG. In other instances, the 1, 5-AG sensor 22 can determine the presence of 1, 5-AG indirectly such that an effect that reflects the 1, 5-AG can be detected and correlated to the presence of 1, 5-AG. The 1, 5-AG sensor 22 can include a working electrode, a counter electrode, and a reference electrode. In some instances, the 1, 5-AG sensor can include a three-electrode cell with three separate electrodes, operating as the working electrode, the counter electrode, and the reference electrode, respectively. In other instances, the 1, 5-AG sensor can include a two-electrode cell with two separate electrode, one electrode operating as the working electrode and the other electrode working as the counter electrode and the reference electrode (e.g., at different times).

The working electrode can include a conductive material. The conductive material can be, for example, a platinum material, which can be a platinum metal, a platinum powder, platinum particles, platinum nanostructures, and/or a platinum-containing conductive ink. When operating indirectly, the working electrode can include or be in contact with a recognition component that interacts (e.g., binds, reacts, or the like) with the 1, 5-AG in the saliva sample to produce a measurable effect, which can be transformed into a measurable signal that can be related to the presence of the 1, 5-AG in the saliva sample. The recognition component can employ biomolecules (e.g., tissue, microorganisms, organelles, cell receptors, enzymes, antibodies, nucleic acids, or the like) to interact with the 1, 5-AG.

The recognition component can be deposited as one or more layers on the surface of the working electrode to cover at least a portion of the surface of the working electrode. The one or more layers can include one or more of functionalized carbon nanotubes or nanoparticles, activated nanoparticles (for example, activated metallic nanoparticles (including one or more transition metals, like gold, platinum, zinc, or copper, for example), and one or more enzymes selective for 1, 5-AG (e.g., pyranose oxidase, L-sorbose oxidase, 1, 5-anhydroglucitol dehydrogenase from the family of oxioreductase enzymes, or any other enzyme that is sensitive to 1, 5-AG). For example, pyranose oxidase enzymes, gold nanoparticles, and carbon nanotubes can be deposited on the working electrode to facilitate the detection of 1, 5-AG based on a product of a reaction catalyzed by the pyranose oxidase enzyme (e.g., H₂O₂). In some instances the functionalized carbon nanotubes, activated nanoparticles, and enzymes can be deposited in a single layer onto the working electrode. In other instances, the functionalized carbon nanotubes can be deposited onto the working electrode as a single layer, while the activated nanoparticles and enzymes can be deposited onto the working electrode as separate layers. It should be noted that the layers can be deposited onto the working electrode with alternative compositions and/or configurations.

The sensing device 12, in some instances, can include additional sensors. For example, as shown in FIG. 3, the sensing device 12 can include the 1, 5-AG sensor 22 and a glucose sensor 32. In some instances, glucose and/or other sugars can interfere with the detection of 1, 5-AG. In indirect sensors, which detect a reaction product from an enzyme-catalyzed reaction and correlate the detected reaction product to the concentration of 1, 5-AG. The reaction product that is detected can be H₂O₂, for example, which can be produced from both the 1, 5-AG reaction and reactions with glucose and/or other sugars. Accordingly, the 1, 5-AG sensor 22 may not be selective for 1, 5-AG. The glucose sensor 32 can be selective for glucose and/or other sugars, without being sensitive to 1, 5-AG. In this instance, the sensing device 12 can send both the indication of 1, 5-AG and glucose/other sugars and the indication of glucose/other sugars to the computing device 14, which can determine the concentration of 1, 5-AG by comparing the two indications and performing simple mathematics.

In some instances, the sensing device 12 can also include additional sensors that can be specific for additional elements. For example, a sensor can be sensitive to the environment (providing environmental data, like temperature, humidity, pressure, and the like) or a condition of the saliva sample (providing sample data, like pH, ionic strength, bacterial load, microorganism load, oxygen content, protein content, reducing agents, and the like). As another example, an additional sensor can be specific to an agent indicating an infection.

Referring again to FIG. 1, the system 10 can also include a computing device 14 that includes a processor 16. For example, the processor 16 can be a microprocessor. In some instances, the computing device 14 can also include a non-transitory memory that can store instructions that are executable by the processor 16. For example, the non-transitory memory can be a random access memory device. However, the non-transitory memory need only be a memory that is not a transitory signal.

The computing device 14 can receive one or more signals from the sensing device 12. The signals can be related to detection of 1, 5-AG and/or additional chemicals in saliva, like glucose. The one or more signals from the sensing device 12 can be transmitted wirelessly and/or according to a wired connection between the sensing device 12 and the computing device 14. The computing device 14 can measure an amount of 1, 5-AG in the saliva sample according to an algorithm. In some instances, the one or more signals can experience signal conditioning 18 before the one or more signals are transmitted to the computing device 14. The signal conditioning 18 can occur within the computing device 14, as illustrated, but can also occur before the one or more signals reach the computing device 14. As one example, the signal conditioning 18 can be a noise removal process. In another example, the signal conditioning 18 can be a normalization of the data between the two signals. Other signal conditioning 18 processes can be undertaken to simplify the analysis of the one or more signals.

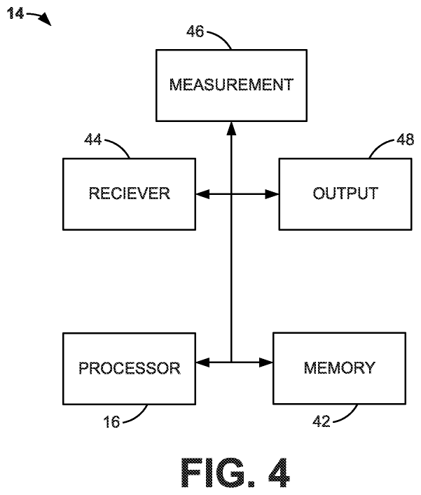

Processing conducted by the computing device 14 is shown in greater detail in FIG. 4. The processor 16 can access the memory 42 to execute instructions related to an algorithm for determining the concentration and/or level of 1, 5-AG in the saliva sample. The steps are implemented as a receiver 44, a measurement unit 46, and an output 48. The receiver 44 can receive one or more signals from sensing unit 12 that include one or more indicators (at least indicating detection of 1, 5-AG). The measurement unit 46 can measure an amount of 1, 5-AG in the saliva sample based on the one or more indicators received. The measurement unit 46 can employ one or more algorithms to determine the amount of 1, 5-AG based on the one or more indicators. For example, the correlation can be direct. As another example, the correlation can be achieved with a predefined mathematical equation. The output 58 can provide an output related to the amount of 1, 5-AG in the saliva sample and/or any additional information in a form that can be perceived through a user's senses. For example, the output can be an audio output, a video output, or the like. In some instances, the detection unit 16 device can be configured with a display unit for a visual display and/or speakers for an audio display.

### IV. Methods

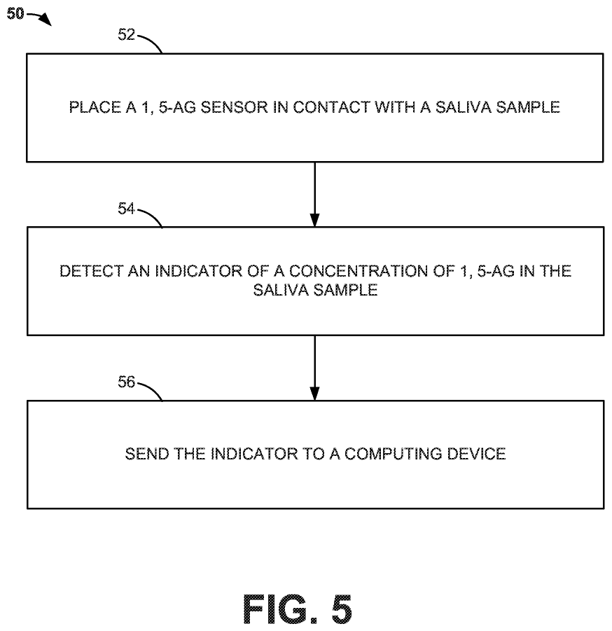

Another aspect of the present disclosure can include methods 50, 60, and 70 (FIGS. 5-7) for detecting the presence of 1, 5-anhydrogluticol (1, 5-AG) in a saliva sample. As an example, the methods 50, 60, and 70 can be executed using the system 10 shown in FIG. 1 and described above. Advantageously, the methods 50, 60, and 70 can be used identify 1, 5-AG levels in saliva, unlike conventional solutions that can detect 1, 5-AG only in blood.

The methods 50, 60, and 70 are illustrated as process flow diagrams with flowchart illustrations. For purposes of simplicity, the methods 50, 60, and 70 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50, 60, and 70.

Referring now to FIG. 5, illustrated is a method 50 for detecting the presence of 1, 5-AG in a saliva sample. As an example, the method 50 can be performed using sensing device 12 of FIG. 1. In some instances, the sensing device 12 can have a single 1, 5-AG sensor 22, as in FIG. 2. In other instances, the sensing device 12 can have a 1, 5-AG sensor 22 and one or more other sensors (e.g., glucose sensor 22) that can be used to determine the presence of 1, 5-AG more precisely.

At Step 52, a 1, 5-AG sensor (e.g., 1, 5-AG sensor 22 of sensing device 12) can be placed in contact with a saliva sample. As an example, the saliva sample can be placed in contact with a predefined portion of the 1, 5-AG sensor. At Step 54, an indicator of a concentration of 1, 5-AG in the saliva sample can be detected. The indicator can be, for example, a current determined according to an electrochemical method (e.g., voltammetry, amperometry, coulometry, or the like). At Step 56, the indicator can be sent to a computing device for further analysis.

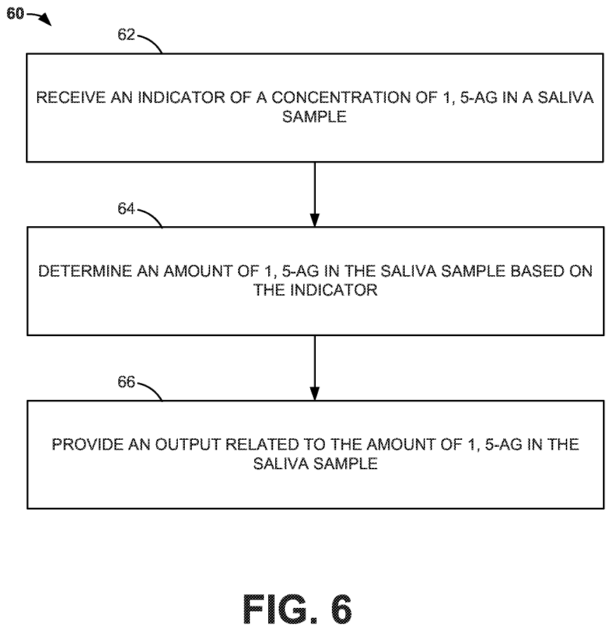

Referring now to FIG. 6, illustrated is a method 60 for determining an amount of 1, 5-AG in a saliva sample. As an example, the method 60 can be performed using the processor 16 of computing device 14 of FIG. 1. At Step 62, an indicator of a concentration of 1, 5-AG in a saliva sample (e.g., a current determined according to an electrochemical method) can be received (e.g., by the processor 16 of computing device 14). Depending on the configuration of the sensing device 12, the processor 16 of the computing device 14 may receive a signal with the indicator from the 1, 5-AG sensor 22 and/or a signal or signals related to the indicator from the 1, 5-AG sensor 22 and another sensor, like glucose sensor 32. After receiving the indicator, in some instances, the indicator (or the signal containing the indicator) can be conditioned or otherwise pre-processed. At Step 64, an amount of 1, 5-AG can be measured based on the indicator. For example, the indicator may directly correlate to the amount of 1, 5-AG. As another example, signals from multiple sensors need to be processed to determine the amount of 1, 5-AG (e.g., by simple mathematics). At Step 66, an output related to the amount of 1, 5-AG can be generated. The output can be, for example, an audio output, a visual output, a digital output including graphical signal, or the like.

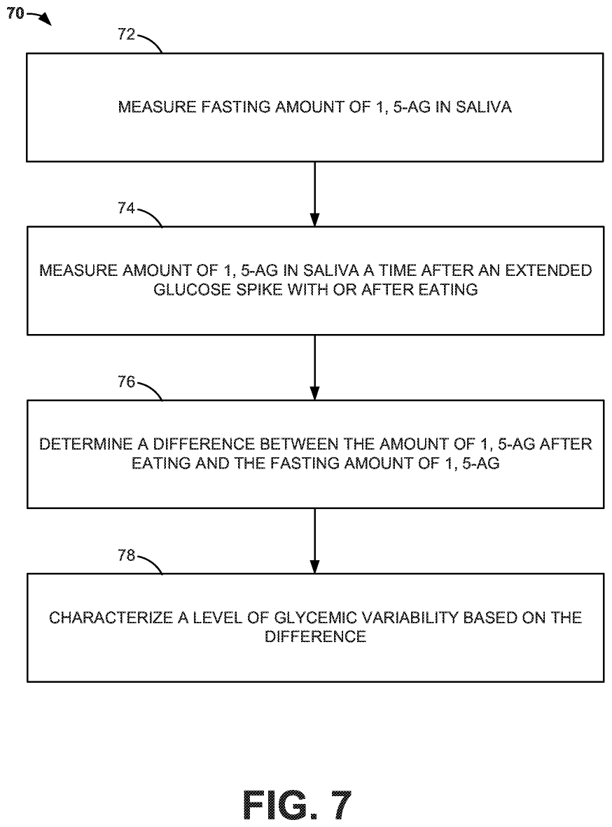

Referring now to FIG. 7, illustrated is a method 70 for determining a level of glycemic variability based on amounts of 1, 5-AG in saliva samples. The method 70 can be performed using the sensing device 12 and the computing device 14 of the system 10 shown in FIG. 1. The sensing device 12 can be as shown in either of FIG. 2 or 3.

At 72, a fasting amount of 1, 5-AG in saliva can be measured (e.g., by placing a fasting saliva sample onto the sensing device 12). At 74, an amount of 1, 5-AG in saliva a time after an extended glucose spike with or after eating can be measured (e.g., by placing a non-fasting saliva sample onto the sensing device 12). For example, the time after the extended glucose spike can be at least 24 hours after eating. At 76, a difference between the amount of 1, 5-AG after eating and the fasting amount of 1, 5-AG can be determined (e.g., by processor 16 of computing device 18 upon receiving the amount of 1, 5-AG after the extended glucose spike and the fasting amount of 1, 5-AG). At 78, a level of glycemic variability can be characterized based on the difference (and in some instances, an output related to the level of glycemic variability can be output). For example, the level of glycemic variability can be variability exhibited (e.g., exhibiting postprandial) or exhibiting minimal glycemic variability. Based on the level of glycemic variability, a diagnosis can be made. For example, someone exhibiting postprandial hyperglycemia may be diagnosed with prediabetes, insulin resistance and metabolic syndrome, or diabetes.

### V. Proposed Configurations

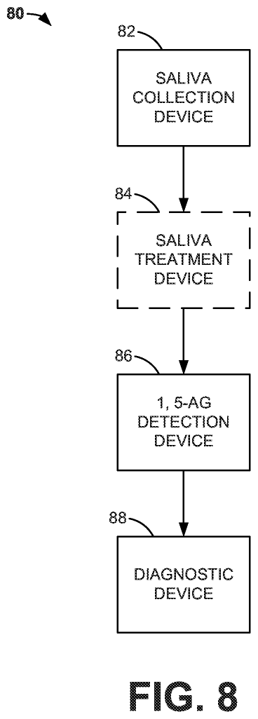

FIG. 8 shows an example group 80 of devices that can be used to detect 1, 5-anhydrogluticol (1, 5-AG) in a saliva sample. In some instances, devices of the group 80 of devices can be implemented as distinct, separate devices. In other instances, at least a portion of the devices can be embodied together within one or more same devices.

The group 80 of devices can include, but is not limited to, a saliva collection device 82, a 1, 5-AG detection device 86 (which may also detect other analytes, like glucose), and a diagnostic device 88. The saliva collection device 82 can collect a volume of saliva for analysis. For example, the saliva can be collected by an absorbent swab. As another example, the sample can be obtained directly as a liquid sample (e.g., via passive drool or spit). The 1, 5-AG detection device 96 can include the 1, 5-AG sensor and, in some instances additional sensors (e.g., a glucose sensor, a contaminant sensor, or the like) (as described above). The sensor(s) of the 1, 5-AG detection device 86 can provide a signal reflecting the detection of the 1, 5-AG and, when there are other sensors, signals from the other sensors reflecting their associated detection. The diagnostic device 88 can process the received signal or signals and determine the amount of 1, 5-AG in the saliva (and additional information) based on the signals. The diagnostic device 88 can also output the determined amount of 1, 5-AG (and/or other information) in a human comprehensible form (e.g., audio, visual, or the like). As an example, the group 80 of devices can also include a sample treatment device 84 that can provide a filter to remove one or more contaminants from the saliva.

### VI. Examples

The following example illustrates that 1, 5-anhydrogluticol (1, 5-AG) can be detected in a saliva sample. Measuring salivary 1, 5-AG (with or without salivary or blood glucose measurement) can be a way to determine whether an individual is diabetic, pre-diabetic, or healthy. Salivary 1, 5-AG can also be measured to screen patients (e.g., in a hospital's intensive care unit) exhibiting hyperglycemia to determine if the hyperglycemia is due to diabetes or due to another medical condition (e.g., an infection, cancer, or the like).

1, 5-AG Sensor Architecture

The 1, 5-AG sensor used in this experiment includes an insulating or semiconducting substrate. In this example, the substrate includes a ceramic insulating substrate. The 1, 5-AG sensor also includes, at least one working electrode, a counter electrode, and a reference electrode. In some instances, the 1, 5-AG sensor can include a three electrode cell with the working electrode, the reference electrode, and the counter electrode (each separate electrodes). In other instances, the 1, 5-AG sensor can include a two electrode cell with the working electrode and the reference electrode/counter electrode (in other words, a single electrode operating as both the counter electrode and the reference electrode). In this example, a three-electrode cell was used. The 1, 5-AG sensor used in this experiment also includes a sample placement area on the surface of the substrate for the saliva to be placed.

The working electrode, counter electrode, and reference electrode are each connected to a detection circuit (e.g., an amperometry circuit, a voltammetry circuit, a coulometry circuit, or other type of electrochemical circuit). An output voltage of the detection circuit provides a measure of the 1, 5-AG concentration in the saliva in the sample placement area. The output voltage is correlated to the 1, 5-AG concentration in the saliva by a function. For example, the output signal can be proportional to the 1, 5-AG concentration or related by some other function, which can be determined using a set of saliva samples having calibrated 1, 5-AG.

1, 5-AG Sensor Construction

The working electrode can be made of a platinum material, such as a platinum metal, a platinum powder, platinum particles, platinum nanostructures, and/or a platinum-containing conductive ink, and/or a carbon material. The following layers were deposited on the working electrode. Single or multi-walled carbon nanotubes (of a size range from 1 nm to 1 μm) were dissolved in deionized water and dried at 30° C. in a curing oven for 30 minutes, which was followed by deposition of biopolymer layer (chitosan 1 mg/mL in acetate buffer). After about 60 minutes of drying, an enzymatic layer mixed with gold activated nanoparticles (from 2 to 200 nm in size) was deposited on top of the biopolymer layer mixed to react with 1, 5-AG. The enzymatic layer included pyranose oxidase (from 0.01 U to 5 U), but could, additionally or alternatively, include L-sorbose oxidase or 1, 5-anhydroglucitol dehydrogenase from the family of oxioreductase enzymes (derived from E. Coli).

Saliva Sample Collection

Saliva samples were collected from non-diabetic individuals through cotton swabs. Each saliva was centrifuged from the cotton swab and then filtered with 0.22 and 0.45 micron syringe filters. Activated charcoal (2 to 20 mg) was used to treat the saliva after filtering.

Glucose and other sugars were found to interfere with detection of 1, 5-AG by the enzyme-based methods described above. For example, glucose and other sugars can react with pyranose oxidase to produce H₂O₂. Accordingly, glucose and other sugars were removed from the saliva samples by pretreating the saliva samples as described above. Accordingly, glucose and other sugars were filtered out the saliva sample before the saliva sample was placed on the 1, 5-AG sensor so that the H₂O₂ produced and the current measured in response to the H₂O₂ were specific to 1, 5-AG.

Sensing 1, 5-AG in Saliva Samples

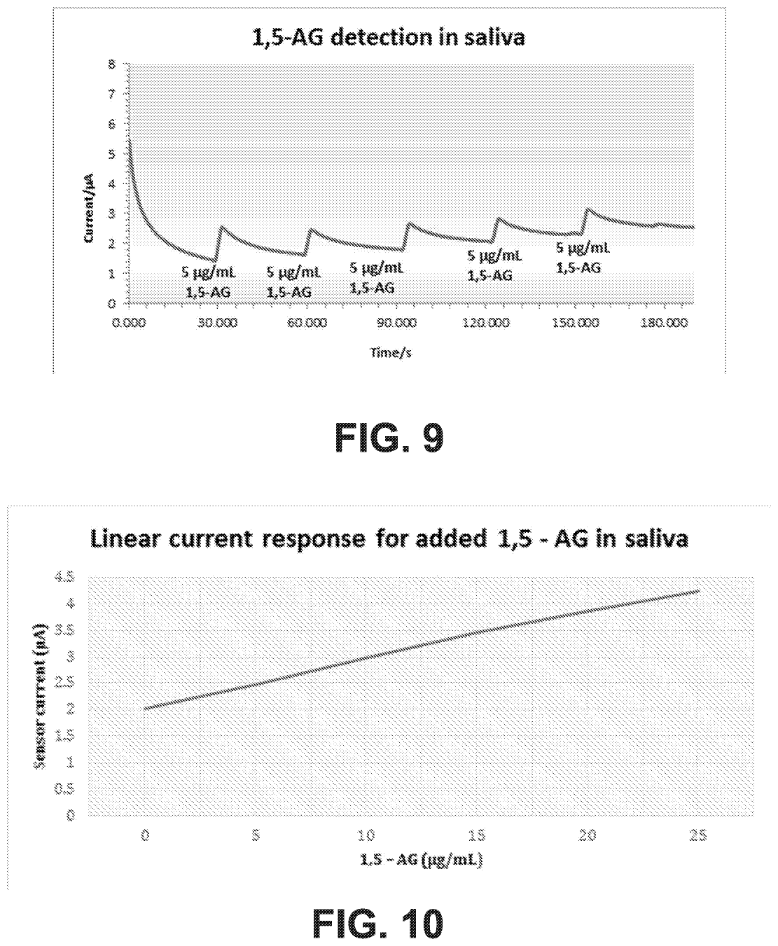

50 μL of a saliva sample was loaded on the 1, 5-AG sensor through a pipette tip. The saliva sample was then spiked with a concentration of 1, 5-AG standard solution ranging from 1 μg/mL to 5 μg/mL every 30 seconds. FIG. 9 shows a current recorded by the 1, 5-AG sensor with spikes corresponding to each 5 μg/mL being added to the saliva sample at 30 second intervals. FIG. 10 shows the linear current response as 1, 5-AG is added to the saliva sample.

Dual Sensor System

An alternate solution to detecting the presence of 1, 5-AG in a saliva sample containing glucose is to use a dual sensor system. The dual sensor system includes a first sensor specific for glucose and a second sensor that can detect 1, 5-AG and glucose. A processing device received a first signal from the first sensor related to the detection of glucose and a second signal from the second sensor related to the detection of 1, 5-AG and glucose. The processing device determines the concentration of 1, 5-AG by comparing the first signal and the second signal (e.g., taking a difference between the first signal and the second signal).

Determining Glycemic Variability

An undiagnosed subject used the 1, 5-AG sensor to measure her salivary 1, 5-AG and her result can out below normal (<10 μg/mL) at 5.5 μg/mL. During random fasting and non-fasting glucose tests, the subject showed normal values (70-80 mg/dL fasting, 95-110 mg/dL non-fasting). An in house A1C test using an A1CNow+ meter indicated the subject had an A1C of 6.5% border line between diabetic and pre-diabetic. Based on these data, the subject was diagnosed as prediabetic and was found to have excursions that were not being caught by fasting and non-fasting glucose levels, but were leading to glycation of proteins.

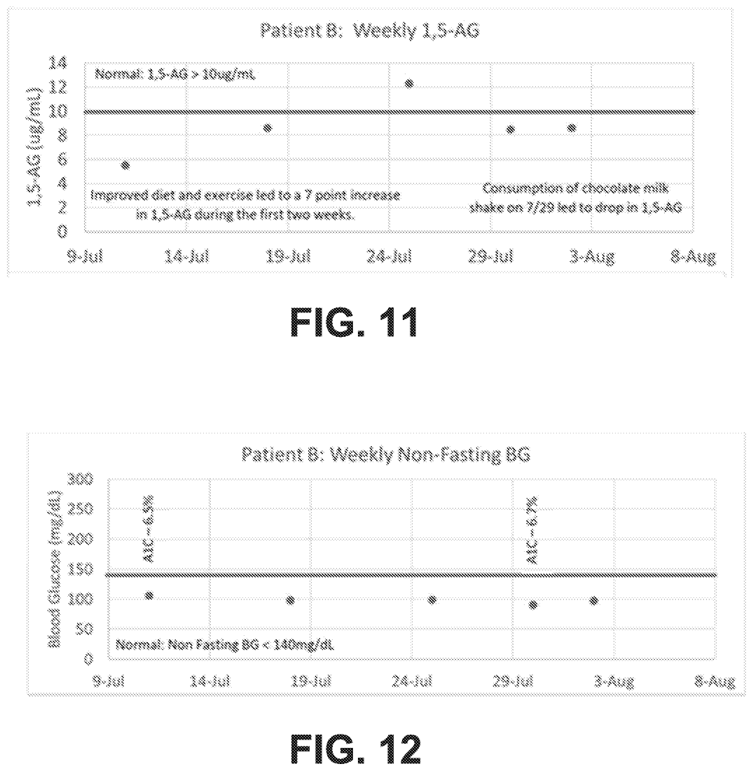

At this time, the subject immediately cut carbohydrate consumption by half (from 50% of diet down to 20-25% of diet) and began taking a walk 20-30 minutes after eating. The impact of the lifestyle changes on the salivary 1, 5-AG measurements are shown in FIGS. 11 and 12, which show the weekly 1, 5-AG measurements with the 1, 5-AG sensor and the weekly non-fasting glucose measurements from 9 Jul. 2018 until 8 Aug. 2018. As shown in FIG. 11, within the first two weeks, the subject saw a 7 point increase in 1, 5-AG (moving from a “low level” or “bad” range—less than 10 μg/mL—to a “high level” or “normal” range—greater than 10 μg/mL). This increase in salivary 1, 5-AG indicates less glucose variability due to better control of postprandial glucose levels.

To verify a drop in 1, 5-AG would be observed with a single hyperglycemic event, the subject consumed a chocolate malt on 29 Jul. 2018 (as shown in FIGS. 11 and 12). The chocolate malt contained over 100 g of carbohydrates. After 48 hours, the subject's 1, 5-AG dropped to 8.5 μg/mL (a drop of 3.8 μg/mL over this 48 hour period). The subject returned to a controlled diet with continued exercise and, after a week and a half, the 1, 5-AG number was back above normal at 10.3 μg/mL. At the same time, a control subject ran the same tests, which indicated that their 1, 5-AG was always above 10 μg/mL (normal).

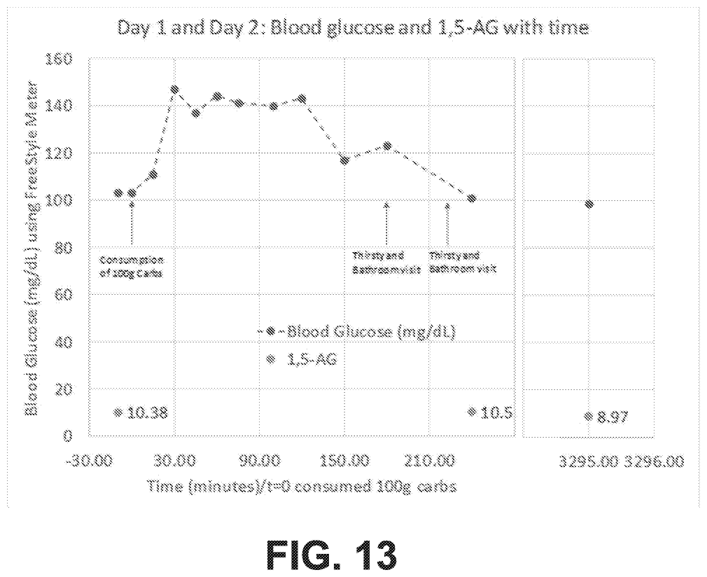

A second time study was done to monitor a hyperglycemic event through measured blood glucose and measured 1, 5-AG (results shown in FIG. 13). The subject arrived fasting in the morning and tested salivary 1, 5-AG with the 1, 5-AG sensor. The subject was determined to have a reading of 10.38 μg/mL of 1, 5-AG. At t=0, the subject consumed 100 g carbohydrates and started testing blood glucose every 15 minutes using a glucometer (Free Style Lite). Within 30 minutes, the blood glucose jumped up to 157 mg/dL, which is considered pre-diabetic. The blood glucose levels stayed around 140 mg/dL (borderline between normal and prediabetic) for almost two hours. Three hours after eating, blood glucose returned to the low 100 s with no change in 1, 5-AG observed. The following morning, 24 hours after consuming the 100 g of carbohydrates, salivary 1, 5-AG was below the normal range of 10 μg/mL, indicating a hyperglycemic event. Based on these results, the 1, 5-AG sensor can detect postprandial hyperglycemia 24 hours after an excursion. With lifestyle changes and better glucose control, especially through diet, salivary 1, 5-AG will reflect better glucose control after two weeks unless another hyperglycemic event occurs causing the value of 1, 5-AG to lower.

From the above description, those skilled in the art will perceive improvements, changes and modifications. Such improvements, changes and modifications are within the skill of one in the art and are intended to be covered by the appended claims.

1. A method comprising:
placing a saliva sample in contact with a sensor device comprising a 1, 5-anhydrogluticol (1, 5-AG) sensor;
detecting, by the 1, 5-AG sensor, an indicator of 1, 5-AG, wherein the indicator of 1, 5-AG is sent to a computing device;
measuring, by the computing device comprising a processor, an amount of 1, 5-AG in the saliva sample based on the indicator of 1, 5-AG,
wherein the amount of 1, 5-AG in the saliva sample is used as a diagnostic indicator.

2. The method of claim 1, further comprising:
placing a fasting saliva sample in contact with the sensor device comprising the 1, 5-AG sensor, wherein the fasting saliva sample corresponds to a patient who is fasting;
detecting, by the 1, 5-AG sensor, a fasting indicator of 1, 5-AG, wherein the fasting indicator of 1, 5-AG is sent to a computing device;
measuring, by the computing device comprising the processor, a fasting amount of 1, 5-AG in the fasting saliva sample based on the fasting indicator of 1, 5-AG;
placing a post-meal saliva sample in contact with the sensor device comprising the 1, 5-AG sensor, wherein the post-meal saliva sample corresponds to the patient a time after eating;
detecting, by the 1, 5-AG sensor, a post-meal indicator of 1, 5-AG, wherein the post-meal indicator of 1, 5-AG is sent to a computing device;
measuring, by the computing device comprising the processor, a post-meal amount of 1, 5-AG in the fasting saliva sample based on the post-meal indicator of 1, 5-AG;
determining a difference between the post-meal amount of 1, 5-AG and the fasting amount of 1, 5-AG;
characterizing the patient as exhibiting glycemic variability or minimal glycemic variability based on the difference.

3. The method of claim 2, wherein the time is at least 24 hours after eating.

4. The method of claim 1, wherein the amount of 1, 5-AG in the saliva sample is determined a time after a patient has experienced an extended glucose spike with or after eating.

5. The method of claim 4, wherein when the amount of 1, 5-AG is less than 10 μg/m L, the patient associated with the saliva sample is experiencing postprandial hyperglycemia.

6. The method of claim 5, wherein the postprandial hyperglycemia is indicative of pre-diabetes, insulin resistance and metabolic syndrome, or diabetes.

7. The method of claim 1, further comprising determining a level of 1, 5-AG in the saliva sample based on the amount of 1, 5-AG in the saliva sample, wherein the level is one of low or high,
wherein the level is used as the diagnostic indicator for the patient associated with the saliva sample.

8. The method of claim 7, wherein when the level is low, the patient associated with the saliva sample is diagnosed as experiencing glycemic variability, and when the level is high, the patient associated with the saliva sample is diagnosed as experiencing minimal glycemic variability.

9. The method of claim 7, wherein the level is low when the concentration of 1, 5-AG is less than 10 μg/mL and the level is high when the concentration of 1, 5-AG is greater than greater than 10 μg/m L.

10. The method of claim 1, wherein the 1, 5-AG sensor employs an electrochemical method to produce a current based on the concentration of 1, 5-AG in the saliva sample, and
wherein the indicator is related to the current.

11. The method of claim 10, wherein the electrochemical method comprises at least one of amperometry, coulometry, and voltammetry.

12. The method of claim 10, wherein the electrochemical method has a sensitivity to detect 1, 5-AG at concentrations from 0 μg/m L to at least 50 μg/m L.

13. A system comprising:
a sensing device comprising a 1, 5-AG sensor configured to be placed in contact with a saliva sample and to detect an indicator of a concentration of 1, 5-AG in the saliva sample; and
a computing device comprising a processing unit configured to:
receive the indicator of the concentration of 1, 5-AG in the saliva sample;
measure an amount of 1, 5-AG in the saliva sample based on the indicator of the concentration of 1, 5-AG in the saliva sample; and
provide an output related to the amount of 1, 5-AG in the saliva sample, wherein the amount of 1, 5-AG in the saliva is a diagnostic indicator.

14. The system of claim 13, wherein the sensing device further comprises a glucose sensor configured to be placed in contact with the saliva sample to detect an indicator of a concentration of glucose in the saliva,
wherein the computing device measures the amount of 1, 5-AG further based om the concentration of glucose in the saliva sample.

15. The system of claim 13, wherein the 1, 5-AG sensor comprises:
a working electrode; and
at least one layer deposited on an outer surface of the working electrode, wherein the at least one layer comprises:
carbon nanotubes;
activated nanoparticles, and
pyranose oxidase enzymes.

16. The system of claim 15, wherein the activated nanoparticles are activated metallic nanoparticles comprising at least one transition metal.

17. The system of claim 16, wherein the at least one transition metal comprises at least one of gold, platinum, zinc, and copper.

18. The system of claim 15, wherein the working electrode comprises a platinum material, wherein the platinum material is at least one of a platinum metal, a platinum powder, platinum particles, platinum nanostructures, and a platinum-containing conductive ink.

19. The system of claim 13, wherein the 1, 5-AG sensor comprises a two electrode cell of:
a first electrode operating as a working electrode; and
a second electrode operating as a counter electrode and a reference electrode.

20. The system if claim 13, wherein the 1, 5-AG sensor comprises a three electrode cell of:
a first electrode operating as a working electrode;
a second electrode operating as a reference electrode; and
a third electrode operating as a counter electrode.
